(19) United States
(12) Patent Application Publication
(10) Pub. No.: US 2024/0164786 A1
(43) Pub. Date: May 23, 2024

(54) LEFT ATRIAL APPENDAGE CLOSURE DEVICE WITH CATHETER-BASED DELIVERY

(71) Applicant: UNIVERSITY OF LOUISVILLE RESEARCH FOUNDATION, INC., Louisville, KY (US)

(72) Inventors: Mark SLAUGHTER, Louisville, KY (US); Guruprasad GIRIDHARAN, Louisville, KY (US); Michael SOBIESKI, Louisville, KY (US); Gretel MONREAL, Louisville, KY (US); Steven KOENIG, Louisville, KY (US); Jorge JIMINEZ, Louisville, KY (US); Landon TOMPKINS, Louisville, KY (US)

(21) Appl. No.: 18/551,043

(22) PCT Filed: Mar. 17, 2022
(86) PCT No.: PCT/US2022/020710

§ 371 Date: Sep. 18, 2023

Related U.S. Application Data
(60) Provisional application No. 63/162,274, filed on Mar. 17, 2021.

(51) Int. Cl. A61B 17/12; A61B 17/00

(52) U.S. Cl. CPC A61B 17/12122; A61B 17/12031; A61B 17/12172; A61B 17/12177; A61B 2017/00367; A61B 2017/00628; A61B 2017/00632; A61B 2017/00867; A61B 2017/12095

(57) ABSTRACT

Instruments and methods are disclosed for left atrial appendage (LAA) closure. An exemplary occluder comprises a lattice framework and an anchor. An exemplary delivery tool provides a catheter-based means of delivery of the occluder to the left atrium and LAA of a heart, such as a human heart, and deployment of the occluder by minimally invasive surgery.

### CROSS-REFERENCE TO RELATED APPLICATIONS

This application claims the benefit of U.S. provisional patent application No. 63/162,274, filed Mar. 17, 2021, the complete contents of which are herein incorporated by reference.

### STATEMENT OF GOVERNMENT INTEREST

This invention was made with government support under Grant No. 1R43HL142337-01 awarded by the National Institutes of Health. The government has certain rights in the invention.

### BACKGROUND

Closure and compression of the left atrial appendage (LAA) has profound benefits in patients that might otherwise suffer a stroke due to nonvalvular atrial fibrillation (NVAF). This is discussed in detail in prior U.S. Pat. Nos. 10,531,878 and 10,898,202, which are both herein incorporated by reference.

Current methods for addressing heart conditions which may lead to stroke include medical therapy, LAA exclusion devices, and LAA occlusion devices.

In the realm of medical therapy, oral anticoagulants, including warfarin, apixaban, edoxaban, clopidogrel, and aspirin, have been used to manage patients with NVAF. Anticoagulation therapy with warfarin has been shown to reduce the risk of stroke by 48% (95% confidence interval (CI), range: 46-51%) to 80% (95% CI, range: 70-91%). However, warfarin dosing must be patient specific and closely monitored, and effectiveness has been linked to patient compliance. Even with close attention to dosing, life-threatening bleeding complications or death occur in 3.09% of warfarin patients each year and between 2.13 and 3.6% for patients using direct anticoagulants. The risk of stroke due to NVAF is greatest in the elderly population, who are also at the highest risk of warfarin complications due to bleeding; thus, nearly 60% of elderly patients with NVAF who are at high risk of stroke are not receiving oral anticoagulant therapy. Further, for every 10% decrease in adherence (not taking medication) there was an increase of 13% in risk of stroke and all-cause mortality. Additionally, while data are emerging from meta-analyses of direct anticoagulants showing efficacy for some that are comparable to that of warfarin, these new anti-coagulants are still plagued by the same issues of lack of patient compliance and severe bleeding complications as warfarin.

LAA exclusion devices such as the Lariat are deployed surgically to close and isolate the LAA from the left atrium (LA) to prevent thrombus. This approach comes with limitations including the need for a surgeon to assist the interventional cardiologist with placement as the procedure is a hybrid thoracotomy and catheter-based procedure, with risks associated with a mini-thoracotomy approach (infection, pain, bleeding). Furthermore, the device may result in incomplete LAA isolation.

LAA occlusion devices are designed to block and/or fill the LAA ostium, which if not completely occluded, can result in leakage and stagnation near the exposed surrounding edges of the LAA orifice increasing the potential risk for thrombogenesis (and stroke). LAA occlusion devices such as the Watchman and Amplatzer are delivered percutaneously via transseptal approach to occlude the LAA from the inside of the LA. These devices, while advantageous due to a minimally-invasive approach, still require the use of anticoagulants to prevent the formation of thrombus until tissue coverage of the device is complete. In addition, these devices may also have design limitations that can result in peri-device leakage, stroke, device-related thrombus, device migration, pericardial effusion, and device fracture. For example, LAA devices with membrane covered frames may only partially fill the LAA chamber (leaving residual volume), thereby producing a large thrombus within the LAA cavity following occlusion, which may produce a corresponding inflammatory response. Peri-device leak, pericardial effusion, and stroke are the most prevalent device-related adverse events for LAA occlusion devices. Peri-device leak has been reported in 12.5% of patients for the Amplatzer, and 20-32% of patients for the Watchman. Furthermore, these devices often do not provide a smooth transition interface between the device and the edge of the striated LAA ostium, leading to areas of blood flow stagnation and thrombogenesis.

### SUMMARY

One aspect of some exemplary embodiments is an improvement to existing left atrial appendage (LAA) closure devices. Another aspect of some exemplary embodiments is a novel catheter-based delivery system for the LAA closure device which permits placement, LAA closure, and, if desired, retrieval from and/or replacement of the LAA closure device in the LAA. For convenience of discussion, this disclosure sometimes uses the term “stroke shield” or “stroke shield system” for the combination of an LAA closure device and a delivery system for the LAA closure device. According to some embodiments, an exemplary stroke shield system comprises an LAA closure device with catheter-based delivery which is configured to prevent strokes in patients with nonvalvular atrial fibrillation (NVAF).

An exemplary stroke shield system comprises a steerable catheter delivery tool and an implantable collapsible occluder (e.g., nitinol reinforced polyethylene terephthalate (PET) umbrella). The collapsible occluder may be sized to be ˜20% (e.g., 18-22%) larger than the LAA orifice and may be curved, e.g., toward the left atrium (LA) wall, to completely cover the LAA orifice regardless of orifice geometry without obstructing the pulmonary veins or mitral valve. The collapsible occluder is deliverable/delivered using a steerable, multi-stage catheter delivery tool (e.g., size 12Fr or smaller) through femoral vein access. The catheter delivery tool is advanced through the venous vasculature into the right atrium (RA), curved using a steerable component to allow for transeptal access into the LA, and then used to anchor and deploy the collapsible occluder to completely cover and occlude the LAA ostium and collapse the LAA to eliminate chamber volume and flow.

Exemplary clinical benefits and technological advantages of the stroke shield system include: (1) complete seal of the LAA (no residual space or flow), (2) smooth endothelialized transition to the LA wall, (3) minimal risk of cardiac tamponade, and (4) catheter-based delivery with the ability to recapture and reposition implant even after full implant deployment. More specifically, advantages of some embodiments may include but are not limited to improving anchoring (migration, strength) and efficacy by reducing the incidence of peri-device flow, pericardial effusion, and cardiac tamponade. Further advantages include steerable control, which can make correct device positioning and deployment via septal access less challenging and require less advanced technical skills than nonsteerable devices.

Some embodiments are designed to completely collapse the LAA eliminating peri-device flow (no residual volume). Some embodiments are designed to promote rapid tissue ingrowth following successful occluder deployment for complete encapsulation of the LAA with endothelialization to form an indistinguishable junction with the atrial wall. In some embodiments a coil anchor provides strong and secure single-point attachment to the LAA free wall to reduce the risk of device migration, while LAA tissue compression is designed to prevent pericardial effusion to minimize the risk of cardiac tamponade. In some embodiments, a single multi-functional catheter-based delivery tool with steerable sheath facilitates occluder placement (angle, location), and enables occluder repositioning and/or retrieval, if needed, even after the occluder has been fully deployed and expanded.

Some embodiments introduce the first LAA mechanical device in the field to combine the technological advantages of LAA exclusion (surgical) and the delivery benefits of occlusion (catheter-based) devices into a single LAA closure procedure by collapsing the LAA with a secure anchoring mechanism to provide a complete seal, eliminate residual volume (no leak), and promote rapid tissue ingrowth and encapsulation (reduce need for prolonged anticoagulation). Exemplary users or operators include but are not limited to interventional cardiologists. Compared with existing devices for LAA surgeries, some embodiments require less variability in device sizing (full orifice coverage independent of LAA perimeter shape), provide tools for accurate deployment (steerable sheath) as well as the ability to reposition, relocate or completely remove the implant, demonstrating ease of use and flexibility, which may lead to broader acceptance by clinical operators with different skill sets. The delivery tool of some embodiments may be the only technology that provides wire access, steerability, and full repositioning or retrieval, thereby improving usability and enabling corrections in cases of size mismatch.

### BRIEF DESCRIPTION OF THE DRAWINGS

FIG. 1 is an exemplary catheter-based delivery tool configured to deliver a collapsible occluder to the left atrial appendage (LAA) of a heart.

FIG. 2A is an enlarged depiction of the exemplary delivery tool.

FIG. 2B is a cross-sectional side profile of the exemplary delivery tool.

FIG. 2C is a cross-sectional side profile of the exemplary delivery tool with slight variations to the handle housings and their connection.

FIG. 2D is an exploded view of the exemplary delivery tool of FIG. 2C.

FIG. 3 shows an exemplary distal bend in a steerable catheter producible with the steerable catheter handle of the exemplary delivery tool.

FIG. 4A is a perspective view of an exemplary collapsible occluder.

FIG. 4B is an end view of the exemplary collapsible occluder.

FIG. 4C is a side view of the exemplary collapsible occluder.

FIG. 4D shows another exemplary collapsible occluder.

FIG. 5A shows an occluder completely collapsed inside a delivery sheath.

FIG. 5B is a photograph of an occluder completely collapsed inside a delivery sheath.

FIG. 5C shows an occluder with only an anchor element deployed from the delivery sheath.

FIG. 5D is a photograph of an occluder with only an anchor element deployed from the delivery sheath.

FIG. 5E shows an occluder completely deployed from a delivery sheath.

FIG. 6A is an exemplary insert with interface elements of an occluder.

FIG. 6B is an exemplary rod system with interface elements of a delivery tool.

FIGS. 7A-7C illustrate exemplary surgical steps for transseptal access and guidewire placement.

FIGS. 8A-8G illustrate exemplary surgical steps for implanting an occluder to close the LAA.

FIGS. 9A-9G illustrate exemplary surgical steps for implanting an occluder to close the LAA.

FIG. 10A is a perspective view of an alternative exemplary delivery tool.

FIG. 10B is a cross-sectional view of the alternative exemplary delivery tool.

FIG. 10C is an exploded view of the alternative exemplary delivery tool.

FIGS. 11A-11F illustrate exemplary surgical steps for implanting an occluder using the delivery tool of FIGS. 10A-10C.

FIGS. 12A-12D are a first exemplary embodiment of interface for coupling/decoupling of an occluder and a delivery tool.

FIG. 13A-13F are a second exemplary embodiment of interface for coupling/decoupling of an occluder and a delivery tool.

FIG. 14A-14D are a third exemplary embodiment of interface for coupling/decoupling of an occluder and a delivery tool.

FIG. 15A-15D are a fourth exemplary embodiment of interface for coupling/decoupling of an occluder and a delivery tool.

FIGS. 16A and 16B are a fifth exemplary embodiment of interface for coupling/decoupling of an occluder and a delivery tool.

FIGS. 17A and 17B are a sixth exemplary embodiment of interface for coupling/decoupling of an occluder and a delivery tool.

FIG. 18 is another exemplary occluder.

FIG. 19 is yet another exemplary occluder.

FIG. 20 illustrates steps of using an exemplary occluder with tissue grasping elements.

FIG. 21A is a photograph of an exemplary occluder.

FIG. 21B is a photograph of an exemplary occluder from a first side and with a fabric covering attached to a lattice framework of the occluder.

FIG. 21C is a photograph of the exemplary occluder of FIG. 21B but from a second side opposite the first side.

FIG. 21D is a plan view of the exemplary occluder of FIG. 21A.

FIG. 21E is a plan view of the exemplary occluder of FIGS. 21B and 21C, with the fabric covering removed.

FIG. 21F is photographs of two exemplary sizes of occluders.

### DETAILED DESCRIPTION

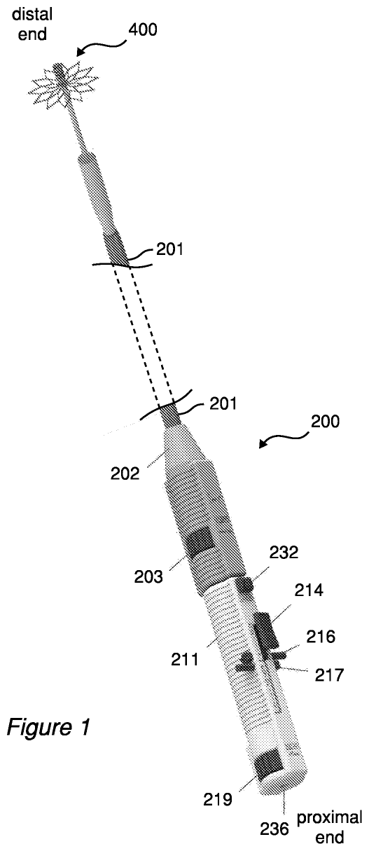

FIG. 1 shows a catheter-based delivery tool 200 configured to deliver an implant, in particular a collapsible occluder 400, via femoral and transeptal access into the left atrial appendage (LAA). The delivery tool 200 comprises a steerable component (outer sheath 201) that allows the tip of the tool to be bent up to 90° inside the right atrium (RA) to allow for atrial septum puncture and insertion. The tip of the delivery tool may be, for example, a delivery sheath 201 (e.g., of size 12 Fr or smaller), which allows for the collapse and concealment of the collapsible occluder 400.

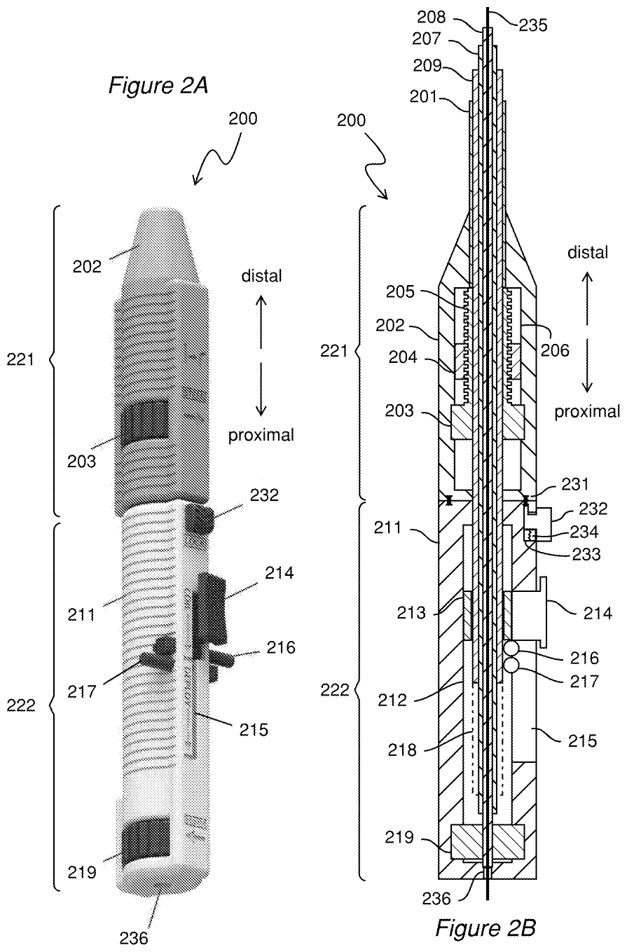

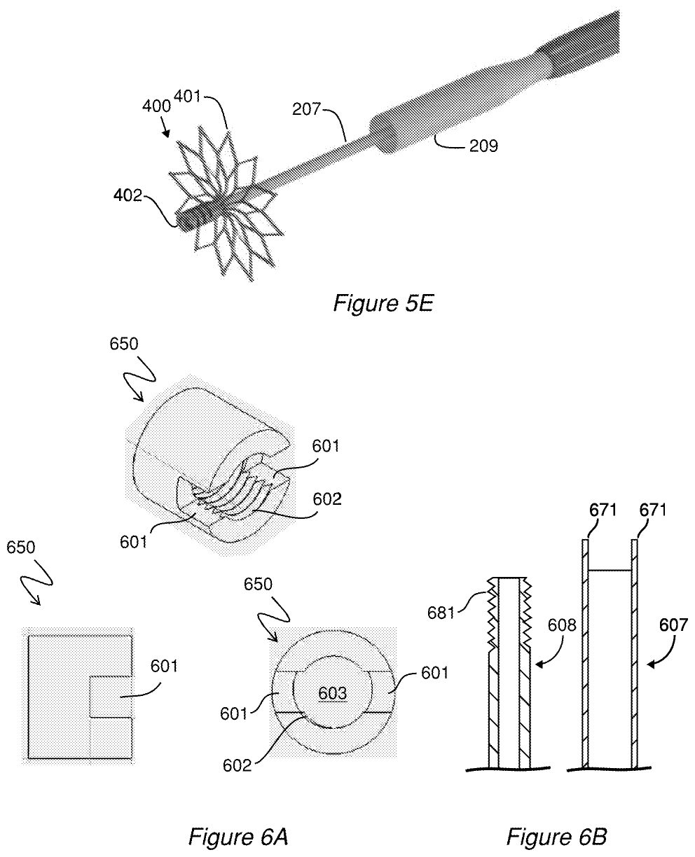

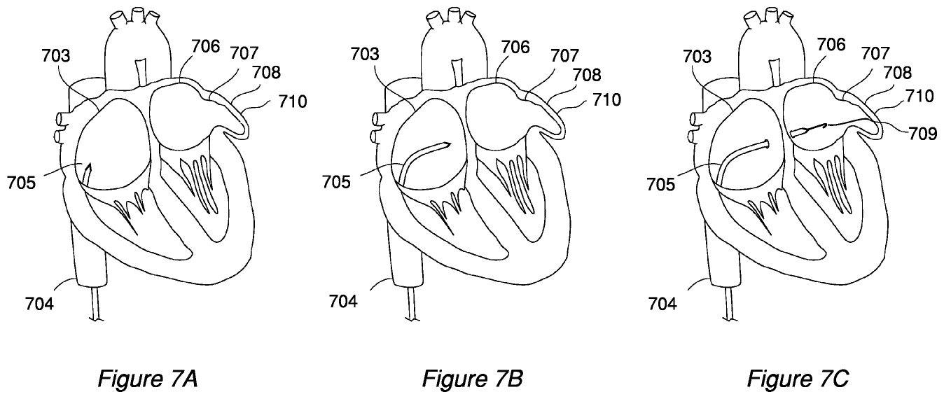

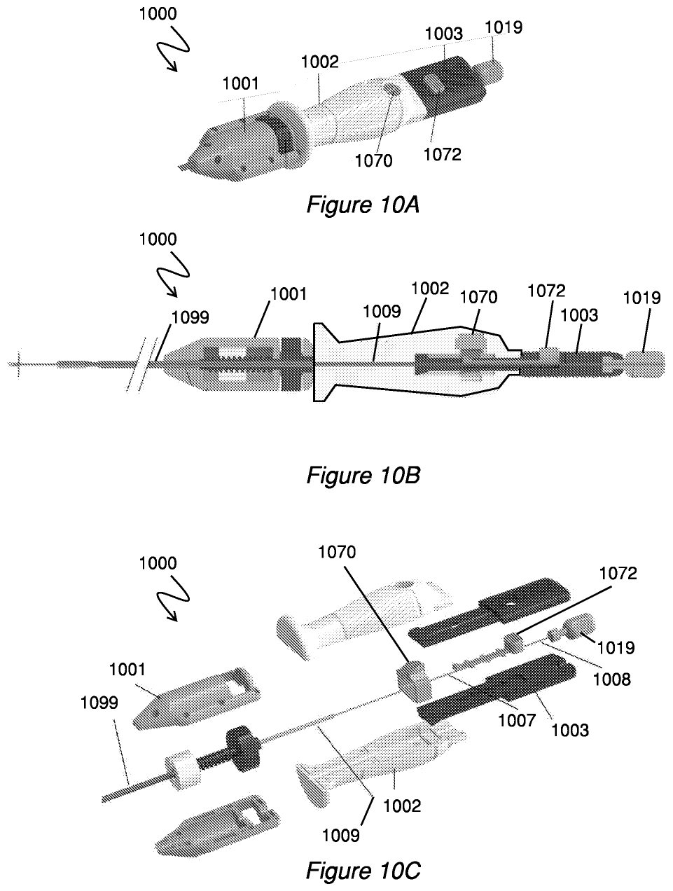

The collapsible occluder 400 may be attached to the delivery tool 200 via an interface which is configured for coupling and decoupling of the occluder 400 and delivery tool 200. The interface may be configured to transfer torque (rotational motion) from the delivery tool 200 to the occluder 400. Internal features of the delivery tool 200 are detailed below in connection with FIGS. 2A and 2B. Aspects of exemplary interfaces between the occluder 400 and delivery tool 200 are detailed below in FIGS. 6A and 7A as well as FIGS. 12A-12D, 13A-13F, 14A-14D, 15A-15D, 16A-16B, and 17A-17B.

The collapsible occluder 400 comprises a coil anchor to secure and collapse the LAA wall and an expanding stent umbrella (e.g., with a circular profile) which is deployable after the anchor is secured to occlude the LAA ostium. The result is closure of the LAA with complete seal (tissue integration) and insubstantial or no residual chamber space (eliminating LAA volume/preventing peri-device leak). The delivery tool 200 gives an operator (e.g., a surgeon) control over each of these stages of delivery and installation.

“Proximal” and “distal” may be used to describe the relative arrangement of various elements. For purposes of this disclosure, something which is “proximal” is nearer the surgeon or other operator during a surgical procedure. Relatedly, something which is “distal” is nearer the patient being operated upon during the surgical procedure. Thus, as depicted in FIG. 1, the LAA occluder 400 is at the distal end of the depicted assembly, and the delivery tool 200 is at the proximal end of the depicted assembly. Reference to a “distal direction” means in the direction of the distal end. Reference to a “proximal direction” means in the direction of the proximal end. Note that this is one non-limiting convention for how “proximal” and “distal” may be used. In some parts of this disclosure or related documentation, these terms may be employed according to other accepted conventions in the medical field. Those of skill in the art will recognize the intended meaning based on the context of use and the supporting figures.

FIG. 2A shows an enlarged depiction of the delivery tool 200 (omitting illustration of catheter outer sheath 201 and other elements inside the sheath 201 for simplicity). FIG. 2B shows a cross-sectional side profile of the delivery tool 200, including illustration of the sheath 201 and elements inside the sheath 201. Note that at the top of FIG. 2B, sheath 201 and elements inside the sheath 201 are truncated but, in practice, extend further, e.g., to an occluder 400 depicted in FIG. 1. FIG. 2C is a cross-sectional side profile of an exemplary delivery tool 200′ which in nearly all respects corresponds with delivery tool 200. Notable exceptions are some variations in the housings of the handles and the connection between the handle components. FIG. 2D is an exploded view of the exemplary delivery tool 200′. Elements which are substantially the same among tools 200 and 200′ share a common label.

The delivery tool 200 comprises one or more controls, sometimes referred to herein as actuators, by which the operator of the tool 200 may trigger or implement various steps or stages of the implantation of the occluder 400 in a patient. In this disclosure, “actuator” may be used to refer to one or more elements of the delivery tool 200 which may, upon being subjected to or receiving a deliberate action of the operator (such as but not limited to pressing, pulling, sliding, and/or twisting/rotating/turning), bring about a corresponding change at the distal end of the assembly in FIG. 1. During an implantation procedure (e.g., LAA closure), the distal end of the assembly in FIG. 1 is inside the patient, whereas the proximal end of the assembly (in particular the parts of the delivery tool 200 depicted in FIG. 2A) are outside the patient's body. Actuators, in many cases, are interfaces at which a surgeon is able to perform an action outside the patient to cause a different but related action inside the patient.

The delivery tool 200 may have one or more handle components, configured for being handled by the operator of the tool. In FIG. 2A, the tool 200 comprises a steerable catheter handle 221 and a delivery handle 222.

The steerable catheter handle 221 is attached to the steerable catheter 201, and these two components may be the outermost components of the delivery tool 200. The handle 221 and catheter 201 may, in essence, be independently operable from all other tool components to allow for free rotation of just the catheter 201 independent of other components within the catheter 201, and conversely, for free rotation of the other components within the catheter 201 independent of the catheter 201. A significant purpose of the steerable catheter 201, and the handle 221 by relation, is to bend the delivery sheath and other components housed partly or entirely within the catheter 201, e.g., up to 90°, inside the right atrium of the heart to allow for straight-shot access to the atrial septum separating the right atrium from the left atrium. In some surgical techniques, alternative methods of access to the left atrium may be employed than by transseptal access from the right atrium. In this case or other cases, the handle 221 and/or catheter 201 may take an alternative configuration or be omitted entirely from the delivery tool 200.

The steerable catheter handle 221 comprises a body 202 and an actuator 203. In this example the actuator 203 is an adjustment wheel which, when rotated, controls deflection of an end/tip portion of the steerable catheter 201 via a braided metal wire embedded in walls of the steerable catheter 201. When the adjustment wheel 203 is turned, a threaded slider 204 mounted on a threaded shaft (e.g., screw) 205 within the body 202 which is attached to the metal wire (the attachment is not visible in FIGS. 2A and 2B) moves axially in either the distal direction or proximal direction, depending on whether the rotation of adjustment wheel 203 is clockwise or counterclockwise. The displacement of slider 203 within a chamber 206 of the body 202 back or forth axially pulls on the internal wire of the catheter 201, which in turn bends the tip of the steerable catheter 201.

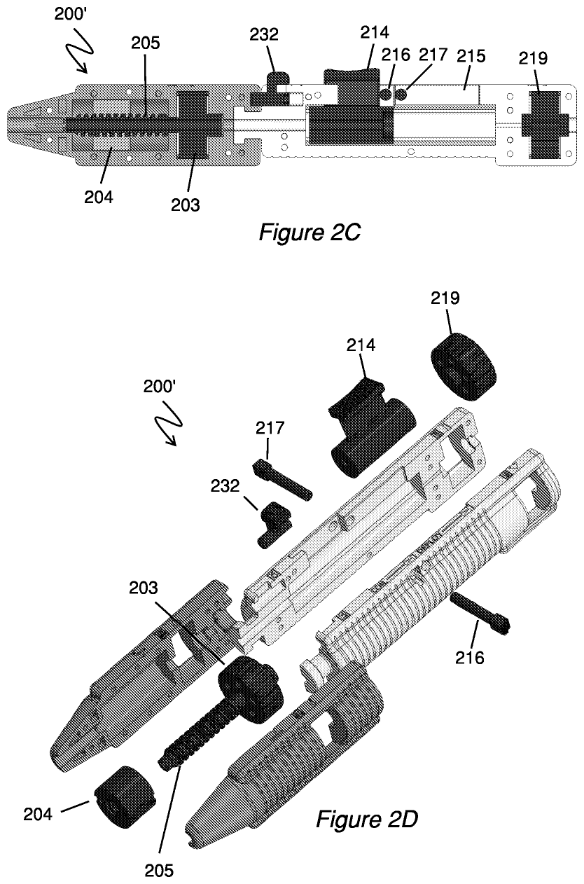

FIG. 3 portrays an exemplary distal bend 300 in catheter 201 producible with the steerable catheter handle 221. Dotted line 301 portrays an original longitudinal axis of symmetry for catheter 201. Dotted line 301 portrays a second longitudinal axis of symmetry for just a distal end portion of the catheter 201 which exists after the bend 300 is created. As already mentioned, the precise angle of bend 300 may vary at any angle from 0° (i.e., no bend) to 90° or more, depending on the amount of rotation supplied to adjustment wheel 203 and, correspondingly, the displacement of slider 203 along shaft 205.

The handle 221 in FIGS. 2A and 2B is but one non-limiting example of a subassembly which permits steering (that is, generally, the changing of the direction of at least the distal end) of catheter 201, and other embodiments may employ alternative steering mechanisms. For example, in some embodiments the embedded braided metal wire of the catheter 201 may be controlled by axial slider buttons on the steerable catheter handle 221, which are slid (translated) back and forth to deflect the steerable catheter 201. Other steering techniques and mechanisms, whether available commercially at the time of this disclosure or in the future, may likewise be employed without leaving the scope of the present technology.

Returning to FIG. 2B, a collapsible occluder and the delivery tool 200 may be coupled (e.g., attached) with one another via interfacing elements of the occluder and delivery tool. Non-limiting examples of specific exemplary interfaces for coupling and decoupling are detailed below in connection with further figures. A variety of different interfaces, however, are actuatable (e.g., to couple, or else to decouple) using a rod system depicted in FIG. 2B.

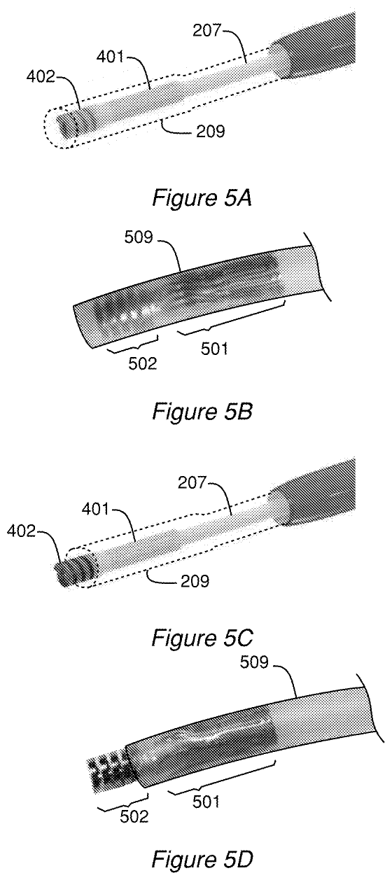

In FIG. 2B, the delivery tool 200 comprises a first rod 207 and a second rod 208. Both may be central rods, e.g., they are aligned with a center longitudinal axis of the delivery tool 201 and the catheter 201. These rods 207 and 208 may hold a collapsible occluder stationary as a delivery sheath 209 is moved relative to the rods and occluder, or vice versa (the rods may move the occluder while the delivery sheath remains stationary). During an exemplary surgical procedure, the rod 207 and/or 208 may hold an occluder at a fixed position while the delivery sheath 209 is retracted to deploy the stent umbrella of the occluder (such a deployment is detailed further below in connection with FIGS. 5A-5D). A rod system such as that depicted by FIG. 2B advantageously permits the recapture of an occluder back into a delivery sheath if device placement needs to be moved or aborted. Note that for purposes of this disclosure, the term “rod” may sometimes imply but does not necessarily require the so-named structure be straight, much less entirely straight. As already discussed above, the catheter 201 is configured to bend elements inside the catheter 201, which include rods 207 and 208, as depicted by FIG. 2B. In many embodiments, rods 207 and 208 will at a minimum be elongate structures.

The delivery handle 222 is so-called for purposes of this discussion because it may be gripped or otherwise handled by an operator and because it comprises one or more actuators relating to the delivery of an occluder to the LAA of a patient. In some embodiments, one or more handle features may be separate and apart from such actuators. FIG. 2B is but one non-limiting example.

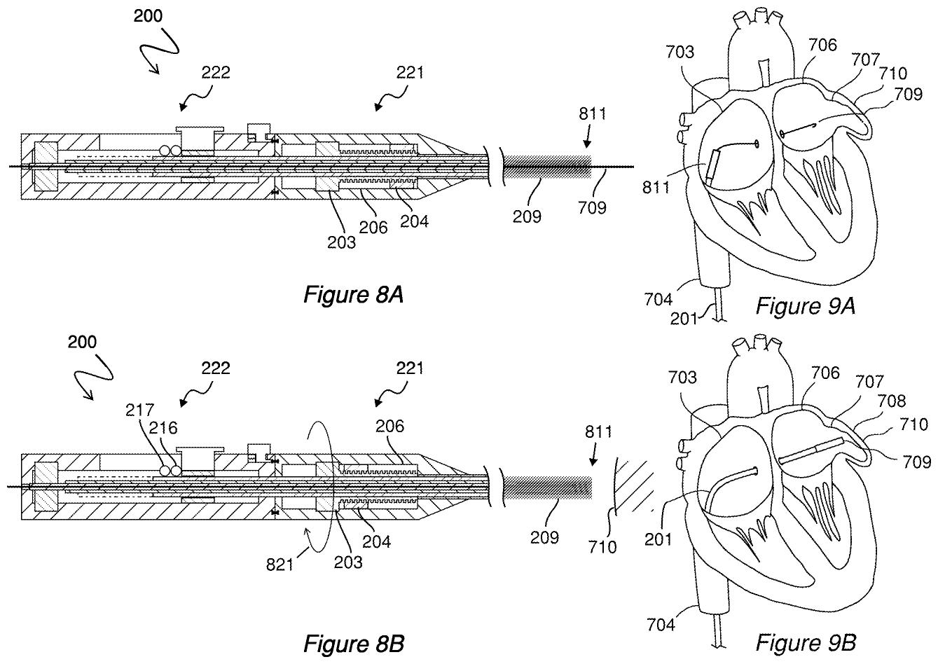

For the sake of introduction, elements illustrated by FIG. 2B will now be identified. Their functions and use in an exemplary surgical method will be discussed further below, in connection with FIGS. 8A-8I. The handle 222 comprises a body 211 in which is a chamber 212. The delivery sheath 209, rod 207, and rod 208 extend from the distal end of the delivery tool 200 into the body 211 and, in particular, the chamber 212. Elements 201, 209, 207, and 208 are substantially coaxially aligned. In different embodiments, sizes (e.g., diameters) of one or more of these elements 201, 209, 207, and 208 may vary from the relative diameters depicted such that gaps or empty space may exist between the outer wall of one element and the inner way of the adjacent element. Both element sizes and element materials are selected to allow acceptably unrestricted movement (e.g., low friction) of elements 201, 209, 207, and 208 relative one another in manners consistent with the exemplary methods detailed in this disclosure.

A delivery sheath mover 213 is configured to grip an external surface of the delivery sheath 209. The mover 213 is moveable along a longitudinal axis (in the distal direction and proximal direction) and slides the delivery sheath 209 in equal measure. The mover 213 is attached to or otherwise a part of an actuator 214, in this case a slider 214. The slider 214 is moveable along a longitudinal axis (in the distal direction and proximal direction) and slides the delivery sheath 209 in equal measure. A slot 215 in the body 211 allows for the actuator 214 to be outside the body 211 but extend into the chamber 212 to grip the delivery sheath 209 with mover 213 inside the chamber 212.

A first lock 216 and a second lock 217 are provided in the slot 215 in the path of the actuator 214. The locks 216 and 217 may also be referred to as stops. They are configured to stop or prevent displacement of the actuator 214, and corresponding movement of the delivery sheath 209 relative to the rods 207 and 208, before such relative movements are desired by the operator. When the operator desires to move the actuator past the locks 216 and 217, the locks are moveable out of the path of the actuator 214 in the slot 215. A dotted line 218 shows the outline of the delivery sheath 209 were it maximally displaced toward the proximal end of the delivery tool 200 by actuating the actuator 214 after removal of both stops 216 and 217.

A rod actuator 219 contacts or otherwise connects to one or both rods 207 and 208 to effect an actuation on the corresponding rod. As illustrated, the rod actuator 219 is a release mechanism, in particular a release wheel, the rotation of which causes the rotation of rod 208.

As will be discussed below, rotation of the handle 222 with respect to the handle 221 (or of the handle 221 with respect to the handle 222) may be desired. Accordingly a connector 231 which connects body 202 of handle 221 and body 211 of handle 222 is configured to permit the relative rotation of either body relative the other body. A handle rotation lock 232 prevents accidental rotation. The lock 232 is slidable within a slot 233 to disengage the lock and permit the relative rotation of the bodies. A spring 234 supplies a return force to urge the lock 232 into the locked position when the operator is not actively maintaining the lock 232 in a disengaged/unlocked position.

A guidewire 235 is able to run through the length of the delivery tool 200. A hole 236 is provided in the body 211 at the proximal end of the delivery tool 200 for this purpose.

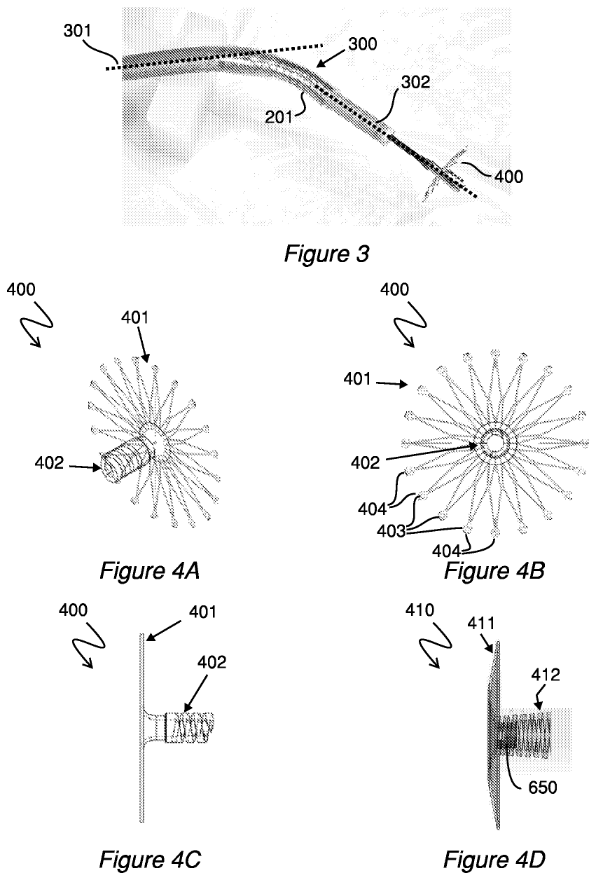

FIGS. 4A, 4B, and 4C show respectively a perspective view, an end view, and a side view of an exemplary collapsible occluder 400. The occluder 400 comprises a lattice framework 401 and an anchor 402. One type of lattice framework frequently referred to herein for ease of discussion is a stent umbrella 401. It should be appreciated that where “stent umbrella” appears in this disclosure, other types of lattices, frameworks, and/or stents which are suitable for covering ostia or orifices (and which may or may not qualify as an “umbrella” configuration) may be used in alternative configurations from those exemplary embodiments which are illustrated.

The stent umbrella 401 is a non-limiting example an occluding portion of the occluder 400. The occluding portion, when in a deployed position, is configured to occlude and provide a seal between a left atrial appendage and a left atrium of a heart (e.g., a human heart, a porcine heart, a mammalian heart, or some other heart). When the occluding portion is in the deployed position, it extends outward to form a substantially flat disc (although in some alternative embodiments some radial curvature may be provided) with the anchor connected at or near the center.

The stent umbrella 401 may be covered with a woven material such as polyethylene terephthalate, also called PET plastic, which sometimes goes by the tradename Dacron. The woven material may be selected or configured to facilitate tissue in-growth and encapsulation. In other embodiments, the umbrella 401 may be covered with an expanded Teflon (ePTFE), animal pericardium, other animal de-cellularized tissue, silk, or other suitable medical fabric or covering to promote tissue ingrowth.

In some embodiments, the occluder 400 includes fabric attachment holes 403 on lattice members at a circumferential periphery of the umbrella shape to which the fabric covering is secured. In some embodiments, the occluder 400 includes rounded stent tips 404 on lattice members at circumferential periphery of the umbrella shape. The fabric covering may also be sewn directly to the stent struts, without the need for attachment holes at the stent strut tips. In some embodiments the fabric may be porous to promote rapid growth and generate a biological seal. In some embodiments, the fabric may be non-porous to seal immediately after implanted. In some embodiments a multi-layered fabric may be used to allow both for rapid seal and texture to promote tissue ingrowth.

The anchor 402 is configured to anchor/secure the occluder 400 to a wall of the LAA. The anchor 402 is proximal to the stent umbrella 401. One exemplary means of producing the anchor 402 is by a helical cut placed in a tube (e.g., of metal or metal alloy such as Nitinol) to form a coil (similar to a cork screw) with a sharpened leading tip. The helical, coiled, and/or spiral nature (depending on the embodiment one or more of these descriptors may apply) of the anchor 402 provides minimal leaks, superior strength, and long-term securing ability. As sample test data of anchor performance, a 2.5-turn coil anchor matching the appearance of FIGS. 4A-4C provided 35 mm² of anchoring surface area with three times the pull-out force of suture in cardiac tissue (coil=15 N, suture=5 N), thereby reducing the risk of device migration or myocardial tear compared to anchoring techniques which exclusively rely on suturing. In addition to its anchoring functionality, the anchor 402 is configured to compress the LAA wall by creating an outward tissue dimple on the external surface of the LAA wall due to radial myocardial compression. Since the anchor 402 constitutes only a single contact point required to collapse the LAA wall and secure the stent umbrella 401, which in turn occludes the ostium of the LAA, the risk of bleeding or tamponade is minimal. The occluder is configured to promote tissue integration by collapsing the LAA orifice and covering all surrounding edges at or near the LAA ostium to completely encapsulate the LAA, thereby helping to minimize peri-device flow.

Exemplary occluders 400 may be manufactured according to a variety of techniques. Following are a few examples. A collapsible occluder may be constructed from a single extruded Nitinol (Nickel-Titanium) tube (exemplary dimensions: 1.6 mm inner diameter, 2.8 mm outer diameter). The stent umbrella is fabricated by grinding one section of the tube to thin the wall thickness, then by using precision laser cutting techniques to carve a lattice framework (stent). This lattice is then expanded to form the Stent Umbrella. The device is then heat treated (annealed with cold water quench) to set the shape of the Stent Umbrella and to activate the super-elastic and shape memory properties of the Nitinol. The opposite end of the tube is cut to form the anchor. In other embodiments, the collapsible occluder may be constructed from multiple parts. For instance, the stent umbrella and anchor components are made from separate tubes and then joined (welded) together to form a singular device.

FIG. 4D shows another occluder 410. Occluder 410 has an occluding portion 411 which may be described as having a shallow bowl or concave/convex disk shape. Occluder 410 further comprises an anchor 412 which is spiraled instead of helical. According to one acceptable meaning of these terms as applied to some embodiments such as that of occluder 410, helical may be used to describe a progressing circular path of constant radius, whereas spiral may be used to describe a progressing circular path of reducing or expanding radius. The occluder 410 comprises an insert 650, discussed in detail below in connection with FIG. 6A. The occluder 400 of FIGS. 4A-4C likewise may include an insert like insert 650, although such an insert is not depicted in FIGS. 4A-4C for simplicity of illustration.

FIGS. 5A-5E illustrate the collapsible nature of some exemplary occluders. It is desirable in many embodiments that an occluder for the LAA be collapsible to render it temporarily in a more compact form suitable for delivery to a region inside the body via a catheter. Accordingly an LAA occlusion surgery may be performed by minimally invasive surgery.

In FIG. 5A, a stent umbrella 401 of an occluder is collapsed and bent inside a delivery sheath 209 (shown transparent with edges marked by broken lines) in such an orientation that does not increase overall device diameter with an increase in (deployed) umbrella radius. Said differently, irrespectively of the radius of different sized occluders, all such different sizes may be fit in the collapsed state inside a delivery sheath 209 of a single size. For instance, the same steerable 12 Fr sheath (or smaller) may be used for a variety of device umbrella sizes (e.g., 21, 25, 30, or 35 mm) to adapt to varying LAA orifice geometries. A single size anchor 402 is suitable for different sized umbrellas 401. A single size and configuration of rod system (comprising at least rod 207) may likewise be used irrespective of different sizes of umbrellas 401.

FIG. 4B shows an actual photo of an anchor 502 and a stent umbrella 501 inside a transparent delivery sheath 509 (the edge of which has a solid borderline added for visibility).

FIG. 5C shows a partially deployed state of the occluder. Here, the anchor 402 has been extended from the distal end of the delivery sheath 209 (alternatively, the delivery sheath 209 is retracted relative to the anchor such that the anchor extends from the distal end of the delivery sheath 209). At the illustrated stage of use, the umbrella 401 is still collapsed and positioned in its entirety within the delivery sheath 209.

FIG. 5D shows an actual photo of the anchor 502 and the stent umbrella 501 inside the transparent delivery sheath 509 (the edge of which has a solid borderline added for visibility), this time with the anchor 502 exposed at the distal end of the sheath 509.

FIG. 5E shows the complete deployment of the stent umbrella 401 after the entire occluder 400 is no longer inside the delivery sheath 209 (either by retracting the delivery sheath 209 off of the occluder 400, or else by moving the occluder 400 out of the end of the delivery sheath 209, or else by a combination of these two relative movements).

The means for achieving collapsibility (and subsequent resumption of deployed shape) of a stent umbrella may vary among embodiments. For instance, the material of the stent umbrella may be chosen and configured such that when exposed to freezing or near-freezing temperatures (e.g., −5° to 5° F.), the stent umbrella may be collapsed back to its original tube shape and placed within the delivery tool delivery sheath. Once the device is exposed to body temperature (e.g., 97°-101°0 F.) and deployed from the distal end of the delivery sheath, the stent umbrella will expand back to its heat-set shape, covering the LAA ostium. In other embodiments, the stent umbrella may instead be heat-treated to be strictly super-elastic; as a result, change in temperature is not needed to deform the umbrella and then return it to its set shape. At body temperature the lattice framework assumes the heat set deployed shape in an absence of restricting external forces (e.g., from a delivery sheath) via material shape memory. Both super-elastic and shape-memory properties are achievable with Nitinol alloys, for example.

FIGS. 6A and 6B introduce elements of an exemplary coupling/decoupling interface between a delivery tool (e.g., delivery tool 200 of FIGS. 2A and 2B) and an occluder (e.g., an occluder 400 of FIGS. 4A, 4B, and 4C). In particular, a rod system of a delivery tool may have one or more features which are configured to interface with one or more features of the occluder.

FIG. 6A depicts a perspective view, side view, and end view of an insert 650 which may be fixed in place within an occluder, e.g., by welding. Alternatively, the body of insert 650 may be material which is integral with the stent umbrella and/or anchor. In either case, FIG. 6A shows interface features of the complete occluder. The interface features of this exemplary embodiment include a threaded hole and one or more notches 601. The hole 603 comprises threading 602. Relatedly, FIG. 6B shows a rod 608 (one exemplary embodiment of rod 208 of FIG. 2B) or with threading 681 configured to be threaded into threading 602. FIG. 6B also shows a rod 607 (one exemplary embodiment of rod 207 of FIG. 2B) with projections 671 configured to fit one apiece into notches 601.

An insert such as insert 650 of FIG. 6A (or at least its interfacing features) may be arranged generally along or symmetrically about the longitudinal center axis of the occluder in some exemplary embodiments. For instance, the insert may be placed at or near the meeting of a stent umbrella and an anchor. Exemplary but non-limiting threading size is M2×0.25. The rod 608, sometimes referred to as a threaded rod for this embodiment, has a matching size to allow attachment and securing of the occluder to the threaded rod of the delivery tool. The insert 640 has two notches 601 (which in alternative embodiments could include one, two, three, four, or more notches) to interface with the rod 607, sometimes referred to as a holder rod for this embodiment, of the delivery tool. The holder rod holds the collapsible occluder stationary via the notched interface while the threaded rod is free to rotate in and out of the threads 602 of the insert 650. The collapsible occluder includes a pass through opening through the entire device to allow for guide wire insertion, tracking, and removal. Exemplary but non limiting sizes for the pass through opening are less than 2 mm (e.g., 1.6 mm). Exemplary guide wires are often in the size range of 0.018-0.035 in. The pass through opening extends longitudinally through the length of the insert 650. As depicted by FIG. 6B, the rods 607 and 608 also have through holes configured for passage of a guidewire.

In other embodiments, threads and notches to interface with the delivery tool may be cut directly into the collapsible occluder tube, eliminating the need for a separate insert part that must be combined with other elements such as by welding during manufacture of the occluder.

FIGS. 7A, 7B, and 7C show exemplary beginning steps to a surgical procedure for LAA occlusion. FIG. 7A depicts accessing a patient's right atrium 703 via the femoral vein 704. FIG. 7B shows advancement of a puncture needle 705 of a standard transseptal access system that may be used to cross the septum and reach the left atrium 706. A dilator (not depicted) may be used during this procedure to enlarge the transseptal puncture if needed or desired. Upon approaching or reaching the ostium (i.e., orifice, opening) 707 to the LAA 708, a guidewire 709 may be deployed, as depicted by FIG. 7C. The guidewire will serve to guide a catheter delivering the occluder so it may be anchored to a tissue wall 710 of the LAA 708. At this stage the LAA may be measured using TEE contrast, for example, injected from the puncture needed 705. The measurements may be used to select one size of occluder from a plurality of different available sizes, e.g., provided in a kit which may be brought into the surgical room and into the operating space if desired. At this point the puncture needle 705 may be removed from the patient while the guidewire 709 remains in place.

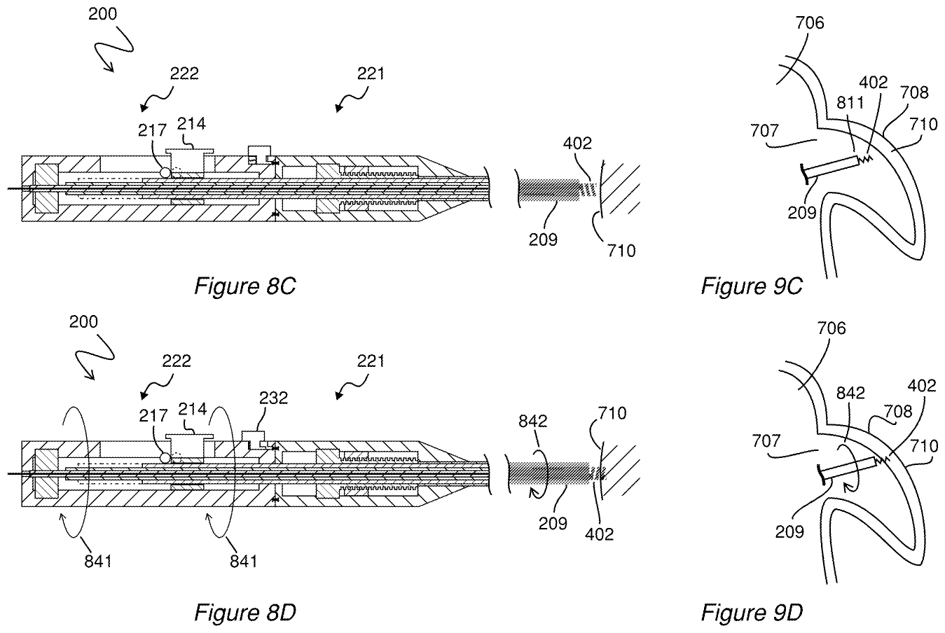

FIGS. 8A-8G show the next series of steps following those of FIGS. 7A-7C. These figures feature the use of the delivery tool 200 (see FIGS. 2A and 2B for corresponding labeling and enlarged depiction of features) together with a close up of the distal end of the delivery tool and its interaction with a tissue wall of the LAA. FIGS. 9A-9G are alternative depictions of the distal end of the delivery tool, including the occluder, and its interactions with the LAA. Each of FIGS. 9A-9G respectively corresponds with the step depicted by FIGS. 8A-8G, respectively.

FIG. 8A shows advancing the distal end 811 of the delivery tool 200 along the guidewire 709.

FIG. 8B shows bending the steerable catheter 209 by rotating (e.g., clockwise) the steering wheel 203. The arrow 821 on the top of the steerable handle 221 indicates the rotation direction of the steering wheel 203. The slider 204 moves within chamber 206 as compared to its position in FIG. 8A.

FIG. 8C shows the coil deployment lock 216 removed (its original position is indicated by broken lines). Pulling back on the delivery sheath slider 214 to stop 217 deploys the anchor 402 from the distal end 811 of the delivery tool 200 so that the anchor 402 is ready to interface with the LAA tissue wall 710. In some embodiments, slight rotation of the delivery sheath 209 (e.g., counterclockwise) may be applied if desired to assist with the deployment.

FIG. 8D shows rotating the delivery handle 222 once the anchor 402 is against the LAA tissue wall 710 at a desired location (e.g., across from the ostium 707). The rotations are for example clockwise according to the illustrated embodiment, as depicted by arrows 841. Arrow 842 shows the corresponding rotation induced in the rod system (contained inside delivery sheath 209) which in turn transfers torque (rotational motion) to the anchor 402, thereby interfacing the occluder 400 with the LAA tissue and anchoring the occluder 400 with the LAA wall 710. The number of revolutions of handle 222 may vary among embodiments, e.g., 1-4 revolutions, or 3 revolutions, for example. The lock button 232 prevents the rotation of the handle 22 prematurely. As depicted by FIG. 8D, the lock button 232 is pulled back (toward the proximal end of the tool 200) to free movement of handles 222 and 221 relative one another at connector 231. During rotation of the handle 222 the steerable handle 221 is held stationary. The lock button 232 is spring loaded by spring 234 so that it will re-lock the handles 221 and 222 relative one another at the end of each revolution. The lock button 232 is pulled back again to rotate for each revolution of handle 222.

FIG. 8E shows the device deployment lock 217 removed (its original position is indicated by broken lines). The delivery sheath slider 214 is retracted further in the proximal direction, e.g., to the maximum displacement permitted by slot 215, to fully deploy the stent umbrella 401. Note that between the steps of FIGS. 8D and 8E, a volume defined by the LAA may be shrunk or collapsed, as depicted by the transition from FIG. 9D to 9E. The shrinking or collapsing of the volume may be achieved in different ways. One exemplary way is by pulling the anchor 402, after it is already secured in the LAA wall 710, toward to the left atrium 706 using the attached rod system. Alternatively, in some embodiments a collapsing of the LAA may be achieved by moving one or more of the anchoring portion and the occluding portion of the implant towards one another. In this case, the anchor and umbrella may be configured to be axially displaceable relative to one another, at least temporarily.

FIG. 8F shows delivery tool release of the occluder. Once occluder placement is confirmed (e.g., by TEE), the occluder 400 is ready to be detached from the delivery tool 200. To completely de-couple the delivery tool 200 and occluder 400 from one another, the release wheel 219 is rotated (e.g., counterclockwise) as indicated by arrow 861 (e.g., approx. 10-12 full revolutions, depending on the thread size of the rod system). Arrow 862 shows the corresponding rotation of rod 208 while holding rod 207 remains stationary and prevents the rotation 862 from transferring to the anchored occluder 400. After sufficient rotations of rod 208, the occluder 400 will be separated from the threaded rod 207.

FIG. 8G shows the delivery tool 200 being removed from the patient. A rotation (e.g., counterclockwise) of actuator 203 is used to unbend elements in the right atrium to complete the instrument withdrawal. The rotation is indicated by arrow 871.

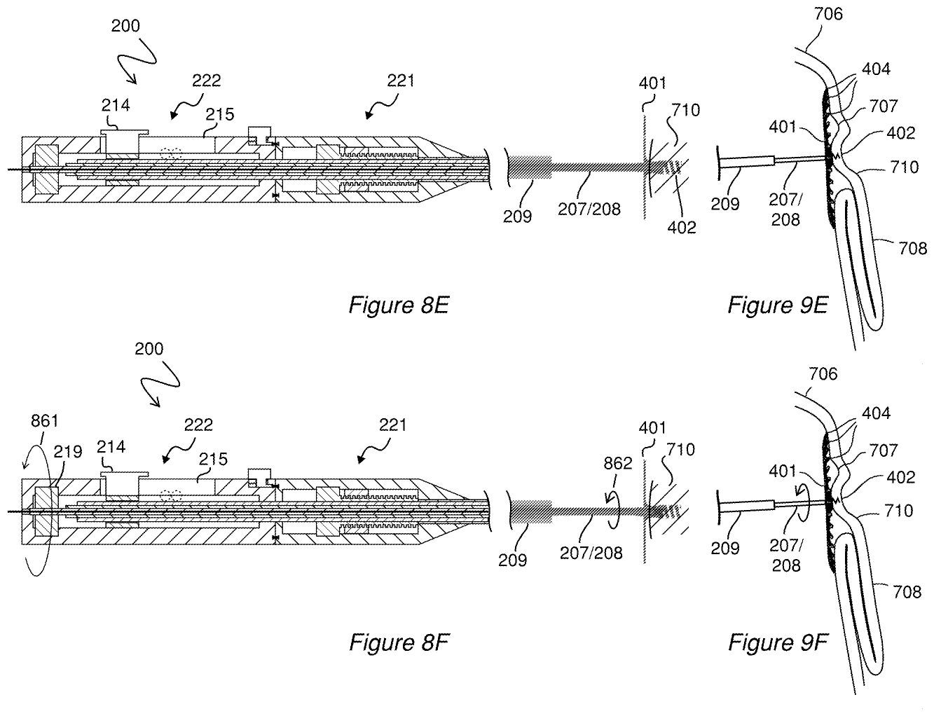

FIGS. 10A-10C show, respectively, a perspective view, a cross-sectional view, and an exploded view of a delivery tool 1000 which shows alternative configurations to the delivery tool 200. Delivery tool 1000 is able to perform the same series of steps as depicted by FIGS. 9A-9G. Delivery tool 1000 differs from delivery tool 200 perhaps most notably with respect to the some of the user interfaces at the handles of the delivery tool.

The delivery tool 1000 allows all actions required of the operator to be control from three main handle components: a steerable catheter handle 1001, a primary handle 1002, and a secondary handle 1003.

The steerable catheter handle 1001 is the distalmost handle and from its end extends the catheter 1099. The primary handle 1001 is attached to the delivery sheath 1009 and houses the anchor deployment button 1070. The secondary handle 1003 is affixed to the primary handle 1001 and slides in and out axially. The secondary handle 1003 is attached to the holder rod 1007 and houses the umbrella deployment button 1072. Attached to the rear of the secondary handle 1003 is the threaded rod knob 1019 which is attached to the threaded rod 1008. When the secondary handle 1003 slides in and out of the primary handle 1001, this in turn allows the holder rod 1007 and threaded rod 1008 to slide in and out of the delivery sheath 1009, and this action is used to deploy the collapsible occluder stent umbrella. The threaded rod knob 1019, when rotated, spins the threaded rod 1008 inside the holder rod 1007, which is held stationary by the secondary handle 1003. This allows the threaded rod 1008 to be threaded in and out of the collapsible occluder insert while the occluder is held stationary via the holder rod interface. The buttons and relative axial displacement of handles in delivery tool 1000 are alternative actuators the those described above for delivery tool 200. Some combination of some actuators from each of these different embodiments may also be used in still further embodiments.

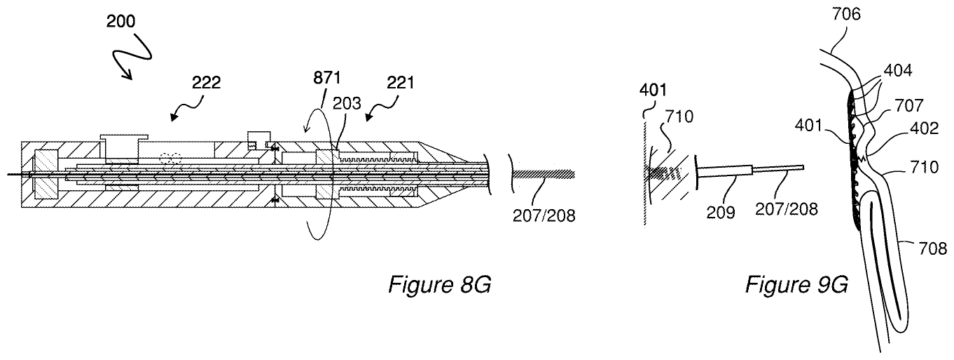

FIGS. 11A-11F illustrated an exemplary sequence of steps for implanting an occluder using a delivery tool 1000. In FIG. 11A, the primary handle 1001 is fully advanced from the secondary handle 1002. In FIG. 11B, the anchor deployment button 1070 is pressed. While the button 1070 is pressed, it allows the secondary handle 1002 to be pushed toward and into the primary handle 1001 until reaching the anchor stop tab 1111 (exemplary displacement of, e.g., 6 mm). In FIG. 11C, the entire delivery tool 1000 is rotated (e.g., clockwise) to screw the coil anchor into LAA tissue. In FIG. 11D, the umbrella deployment button 1072 is pressed. While the button 1072 is held down, the secondary handle 1002 is able to be pushed all the way forward to its maximum displacement relative the primary handle 1001 (e.g., approx. 45 mm). In FIG. 11E, after implant placement is confirmed (e.g., by TEE), the threaded rod knob 1019 is rotated (e.g., counterclockwise) until the collapsible occluder is released. In FIG. 11F, once the collapsible occluder is released, the secondary handle 1002 is retracted to resheath the rods 1008 and 1007 for delivery tool removal from the patient.

FIGS. 12A, 13A, 14A, 15A, 16A, and 17A show several alternative interfaces for the coupling/decoupling of an occluder and a delivery tool, in particular a rod system of that delivery tool. For the most part the depictions are cross-sectional views through longitudinal centerlines of the elements, as indicated by the cross-hatching. Generally, these interfaces are configured to permit various types of force transmissions to the occluder (i.e., the implant for an LAA closure surgical procedure) from the delivery tool on the basis of operator inputs or activity at the handle (or handles) of the delivery tool. Generally, such force transmissions may include but are not necessarily limited to pushing, pulling, and turning (transferring torque to) the occluder using the rod system of the delivery tool. Pushing and pulling generally refer to translational forces, e.g., in the distal direction or in the proximal direction respectively, typically along or approximately along a longitudinal center axis, e.g., of a catheter or delivery sheath of the system. Turning, rotating, twisting, or torquing generally refers to rotational forces about or approximately about a longitudinal center axis, e.g., of the catheter, delivery sheath, one or more rods, and/or occluder of the system. It is furthermore noted that parts of the occluder and parts of the delivery tool (e.g., parts of the rod system) may be collectively referred to as an interface. In addition, the parts of the occluder may be regarded as a first interface, and the parts of the delivery tool may be regarded as a second interface that interacts with the first interface.

The illustrated interfaces are non-limiting examples of different configurations. In some embodiments, the interface may comprise threading or screw-nut attachments (e.g., see interfaces 1200 and 1300). In some embodiments, the interface may comprise deformable or elastic parts such as protrusions, the positions of which correspond with locked or unlocked states between an occluder and the delivery tool (e.g., see interfaces 1400 and 1500). In some embodiments, the interface may comprise a bayonet or reverse bayonet style mount or lock (e.g., see interfaces 1600 and 1700). In some embodiments, the rod system of the delivery tool comprises at least two rods (e.g., see interfaces 1200, 1300, 1400, and 1500). In such cases the rods, in an assembled state of use, may be coaxially aligned and nestable one inside the other. In some embodiments, the rod system may have only a single rod (e.g., see interfaces 1600 and 1700). For convenience of illustration and discussion, elements of the implant (the occluder) are described as being part of an insert. As previously discussed, manufacturing of an insert and subsequently installing it, e.g. by welding, into an occluder centered with the anchor and umbrella is acceptable for some embodiments. However, some embodiments may be manufactured using techniques which do not require a separate insert. Features described as being part of an insert may therefore be features incorporated directly into the occluder structure material, e.g., at or near the juncture of an anchor and stent umbrella of an occluder.

FIG. 12A shows an interface 1200 that comprises an insert 650 (previously introduced in FIG. 6A) and rods 607 and 608 (previously introduced in FIG. 6B). The rods are sized and shaped such that (inner) rod 608 fits inside of a through hole or cavity of (outer) rod 607. The prongs/projections 671 fit into slots 601 of the insert 650. (Screw) threads 681 of rod 608 are sized to fit with the threads 602 of hole 603 of the insert 650. Torque is transferable from either the projections 671 to the notches 601 or the screw threads 681 to threads 602.

FIG. 12B show the interface 1200 with maximum coupling. FIG. 12C shows the result of holding the inset 650 (and thereby the occluder of which it is a part, not shown) with rod 607 while turning the rod 608 to disconnect rod 608 from the inset 650. FIG. 12D shows the withdrawal of both rods 607 and 608 from the inset 650.

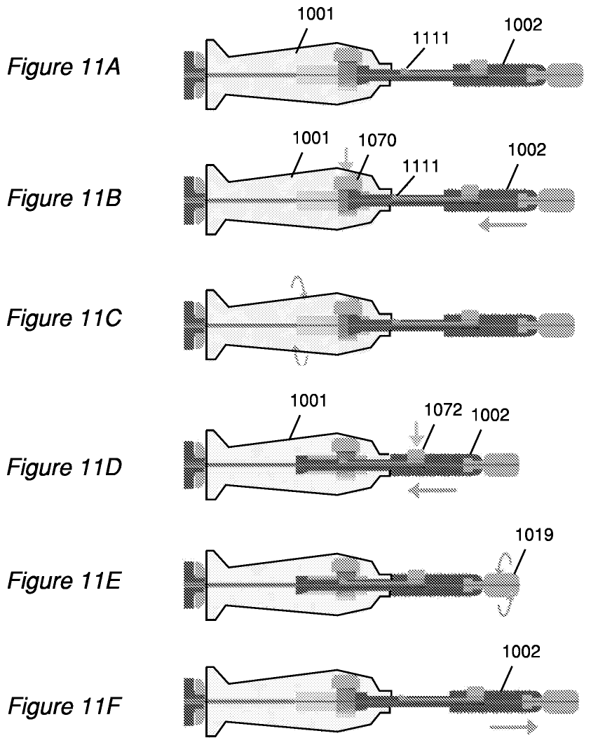

FIG. 13A shows an interface 1300 similar to interface 1200 but with swapped functional roles for inner and outer rods. In interface 1300, the outer rod 1381 has threading 1381, and the inner rod 1371 has one or more projections 1371. The insert 1350 has a notch, gap, or cavity 1301 configured to receive the projections 1371. Torque is transferable from the projections 1371 to the cavity 1301 in much the same manner as a flat head screwdriver transfers torque to the head of a wood screw. FIG. 13F shows a view of the end of the rod 1308 at the end with projection(s) 1371. Relatedly, FIG. 13E shows a view of the end of insert 1350 at the end towards which the threads 1302 open.

FIG. 13B shows the interface 1300 with maximum coupling. FIG. 13C shows the result of holding the inset 1350 (and thereby the occluder of which it is a part, not shown) with rod 1308 while turning the rod 1307 to disconnect rod 1307 from the inset 1350. FIG. 13D shows the withdrawal of both rods 1307 and 1308 from the inset 1350.

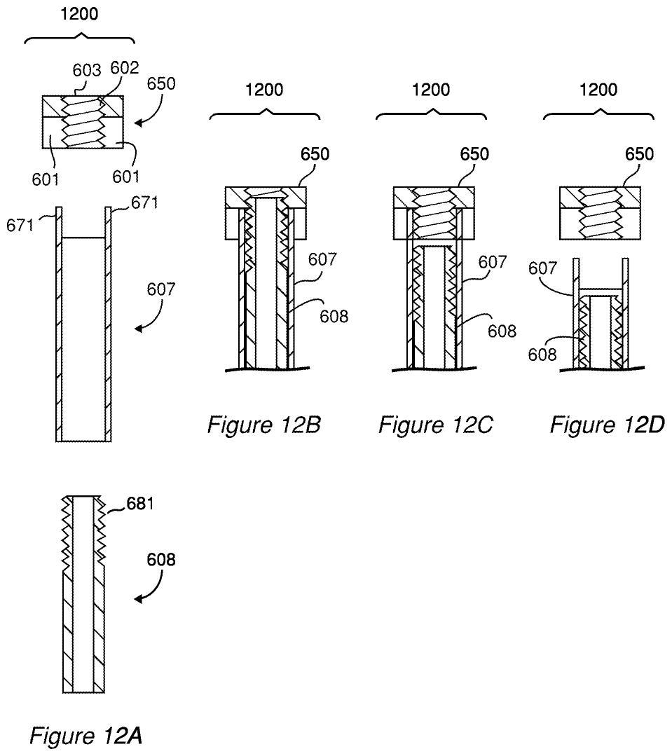

FIG. 14A shows an interface 1400 that comprises an insert 1450, rod 1407, and rod 1408. The interface 1400 has elastically deformable projections 1440 (three are depicted, but one, two, three, or more may be used in alternative configurations) at an end of rod 1407, which in this case is an “outer” rod. Each projection comprises an arm 1441 and a secondary projection, e.g., radial nub 1442. FIG. 14A depicts the relaxed state of the projections 1440. In the relaxed state, the rod 1407 can freely slide into the cavity 1403 of insert 1450. Rod 1408 is slidable into a through hole of rod 1407. Rod 1408 is sized such that when its distal end reaches the projections 1440, it forces the projections 1440 radially outward. Notches or cavities 1401 within insert 1450 are sized and positioned such that the nubs 1442 are received in the notches 1401 when the rod 1408 maximally deforms the projections 1440 from their relaxed positions. In their maximally deformed positions, the projections 1440 with their nubs 1442 are locked into a position within the insert 1450 from which withdrawal of the rod 1407 from the insert 1450 is not possible. In this state (depicted by FIG. 14B), the rod 1407 is capable of transferring axial forces as well as rotation forces to the insert 1450. In other words, the rod 1407 is capable of pushing, pulling, and transferring torque to the insert 1450. In this configuration, the rod 1408 may serve only the unitary purpose of locking and unlocking the rod 1407 to/from the insert 1450.

FIG. 14B shows the interface 1400 with maximum coupling. The nubs 1442 are displaced into notches 1401 by the presence of rod 1408 inside rod 1407 at the longitudinal position of the projections 1440. FIG. 14C shows the rod 1408 withdrawn from the longitudinal position of the projections 1440. As a result, the projections 1440 have elastically returned to their relaxed position, in which the nubs 1442 are not positioned in the notches 1401. In this state, rod 1407 is free to move longitudinally from the cavity 1403, as depicted by FIG. 14D.

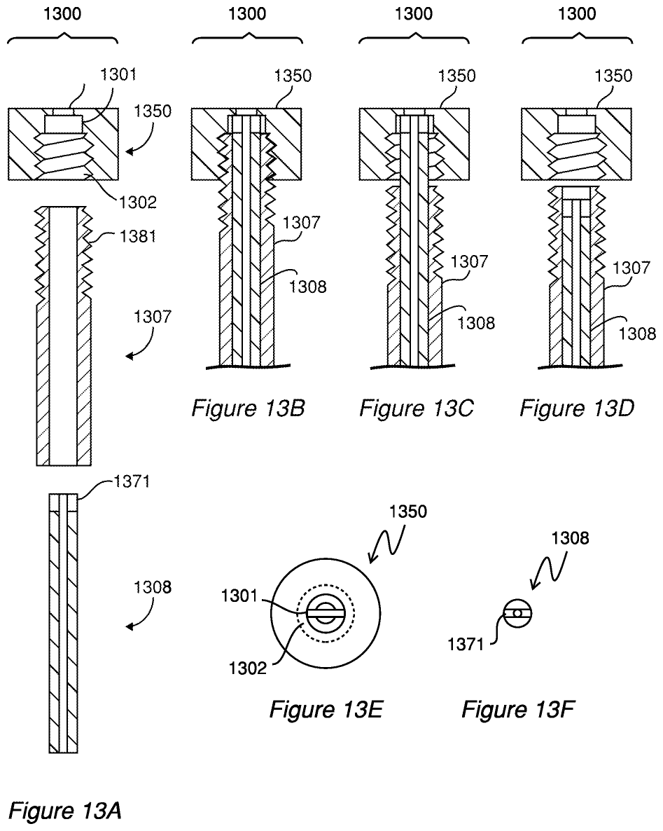

FIG. 15A shows an interface 1500 similar to interface 1400 but with swapped functional roles for inner and outer rods. The interface 1500 comprises an insert 1550, rod 1507, and rod 1508. The interface 1500 has elastically deformable projections 1540 (three are depicted, but one, two, three, or more may be used in alternative configurations) at an end of rod 1507, which in this case is an “inner” rod. Each projection comprises an arm 1541 and a secondary projection, e.g., radial nub 1542. In contrast to inserts of above-described embodiments, all of which may be described as “male” type connectors, insert 1550 may be more aptly described as a “female” type connector. FIG. 15A depicts the relaxed state of the projections 1540. In the relaxed state, the rod 1507 can freely slide over the insert 1550. Rod 1508 (in this case an “outer” rod) is slidable over rod 1507. Rod 1508 is sized such that when its distal end reaches the projections 1540, it forces the projections 1540 radially inward. Notches or cavities 1501 within insert 1550 are sized and positioned such that the nubs 1542 are received in the notches 1501 when the rod 1508 maximally deforms the projections 1540 from their relaxed positions. In their maximally deformed positions, the projections 1540 with their nubs 1542 are locked into a position within the insert 1550. Withdrawal of the rod 1507 from the insert 1550 is not possible while the rod 1508 remains at a longitudinal position corresponding with the projections 1540. In this state (depicted by FIG. 15B), the rod 1507 is capable of transferring axial forces as well as rotation forces to the insert 1550. In other words, the rod 1507 is capable of pushing, pulling, and transferring torque to the insert 1550. In this configuration, the rod 1508 may serve only the unitary purpose of locking and unlocking the rod 1507 to/from the insert 1550.

FIG. 15B shows the interface 1500 with maximum coupling. The nubs 1542 are displaced into notches 1501 by the presence of rod 1508 over rod 1507 at the longitudinal position of the projections 1540. FIG. 14C shows the rod 1508 withdrawn from the longitudinal position of the projections 1540. As a result, the projections 1540 have elastically returned to their relaxed positions, in which the nubs 1542 are not positioned in the notches 1501. In this state, rod 1507 is free to move longitudinally from the insert 1550, as depicted by FIG. 15D.

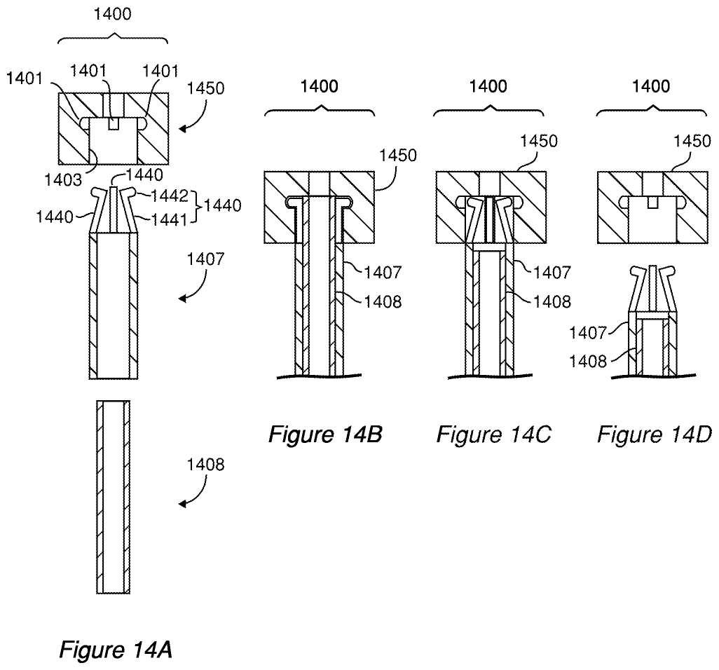

FIG. 16A shows an interface 1600 which comprises a bayonet style connection. This style of connection is but one example by which a rod system comprising or consisting of a single rod—not two rods as in the embodiments discussed above—may be sufficient for allowing coupling/decoupling of delivery tool and occluder, without loss of the ability to push, pull, and turn (transfer torque) the occluder using the rod system of the delivery tool. Rod 1607 comprises radial projections 1671 at or near the distal end of the rod 1607. Two projections 1671 are depicted, but embodiments may have one, two, three, or more than three projections 1671. FIG. 16B shows the rod 1607 rotated 90 degrees relative to the depiction of rod 1607 in FIG. 16A. The insert 1650 has slots, grooves, or notches 1601 configured to receive respective ones of the projections 1671. The grooves may be shaped differently for different embodiments. Generally, however, the grooves and projections cause a rotation of the rod 1607 relative the insert (or a rotation of the insert relative the rod) as the rod 1607 is inserted into the insert 1650. Along the groove, e.g., at the end of the groove, the groove may have a “seat” in which the projections 1671 have a more stable position than in other positions of the groove.

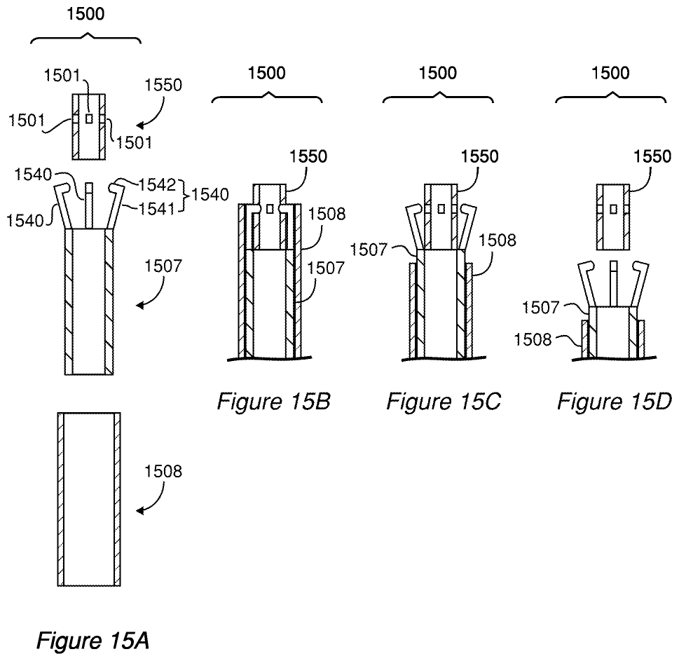

FIG. 17A shows an interface 1700 which comprises a reverse bayonet style connection. This style of connection is but one further example by which a rod system comprising or consisting of a single rod—not two rods as in the embodiments discussed above—may be sufficient for allowing coupling/decoupling of delivery tool and occluder, without loss of the ability to push, pull, and turn (transfer torque) the occluder using the rod system of the delivery tool. Insert 1750 comprises radial projections 1771 at or near the proximal end of the insert 1750. Two projections 1771 are depicted, but embodiments may have one, two, three, or more than three projections 1771. FIG. 16B shows the insert 1750 rotated 90 degrees relative to the depiction of insert 1750 in FIG. 17A. The rod 1707 has slots, grooves, or notches 1701 configured to receive respective ones of the projections 1771. The grooves may be shaped differently for different embodiments. Generally, however, the grooves cause a rotation of the rod 1707 relative the insert (or a rotation of the insert relative the rod) as the rod 1707 is inserted into the insert 1750. Along the groove, e.g., at the end of the groove, the groove may have a “seat” in which the projections 1771 have a more stable position than in other positions of the groove.

Typically, exemplary occluder anchors are securely anchored into the LAA free wall without perforation (no cardiac effusion). However, for some patients or with some embodiments, a potential risk remains for over-torquing during implant that may cause tissue damage. To reduce this potential risk, exemplary occluders and/or exemplary delivery tools may comprise a torque limiting device configured to set an upper limit/ceiling to the amount of torque transferable from the rod system to the occluder.

In some exemplary embodiments, mechanical, chemical, or other means may be used to bend the tissue before delivery of an anchoring element. Bending is used to increase the depth of tissue into which the anchor is to be delivered. In some embodiments the bending element and anchoring elements are delivered from the same side of the tissue wall to be treated, in other embodiments the anchoring and bending elements are delivered from opposing surfaces of the tissue wall.

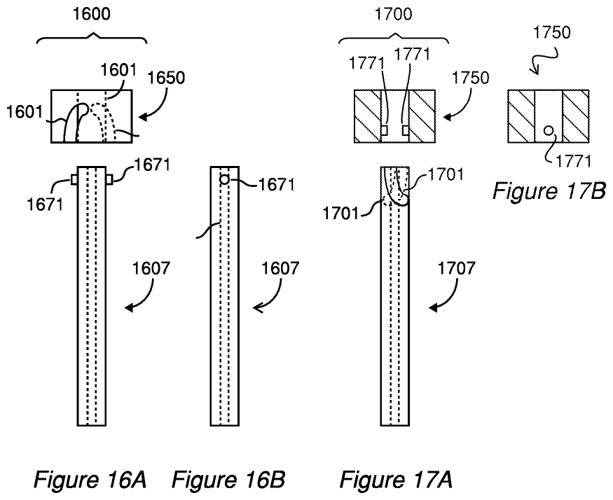

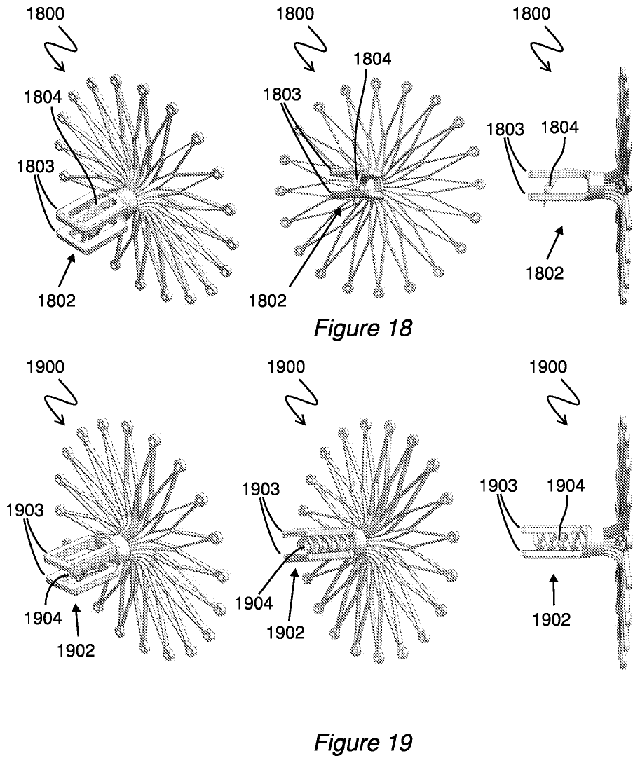

FIGS. 18 and 19 present alternative anchor configurations to those already presented in FIGS. 4A, 4B, 4C, and 4D. FIG. 18 presents an anchor 1802 of an occluder 1800, and FIG. 19 presents an anchor 1902 of an occluder 1900. In both occluders, the anchor incorporates arms or stabilization elements, in particular two (a pair of) jaws 1803 or 1903 (e.g., of Nitinol) which are configured to be hinged open and used to capture a significant amount of tissue of the LAA wall between the jaws. Collecting tissue in this way essentially increases the wall thickness of the LAA tissue, providing more surface area for the primary anchor element (e.g., a curved spike 1804 or coil/spiral 1904) and helping to ensure that the primary anchor element does not advance “too far” into the LAA wall and risk perforating the other side of the tissue wall. An exemplary pair of moveable jaws may be secured to an insert, such as any of those disclosed above, or to an occluder at or near the interface features of the occluder.

In embodiments where a bending element is used to increase the tissue wall depth to be engaged with the anchoring device, the delivery tool may include a mechanism to control the position of the bending element or an engaging mechanism which allows for stabilizing the wall while the bending element generates the change in tissue geometry which is then used for increased depth in the anchor.

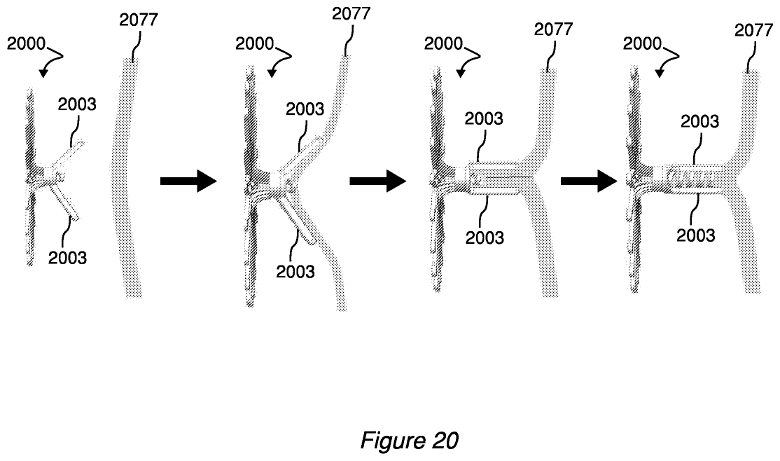

FIG. 20 illustrates the functioning of an anchor that comprises a pair of jaws. In some embodiments, as shown in FIG. 20, the arms or stabilization elements 2003 of the occluder 2000 are configured to bend the LAA tissue 2077 and effectively increase the wall thickness. The elements 2003, which may be characterized as jaws, are directly part of the collapsible occluder implant 2000 itself. In alternative embodiments, the elements 2003 used to bend the tissue in the desired configuration may instead be parts of the delivery tool.

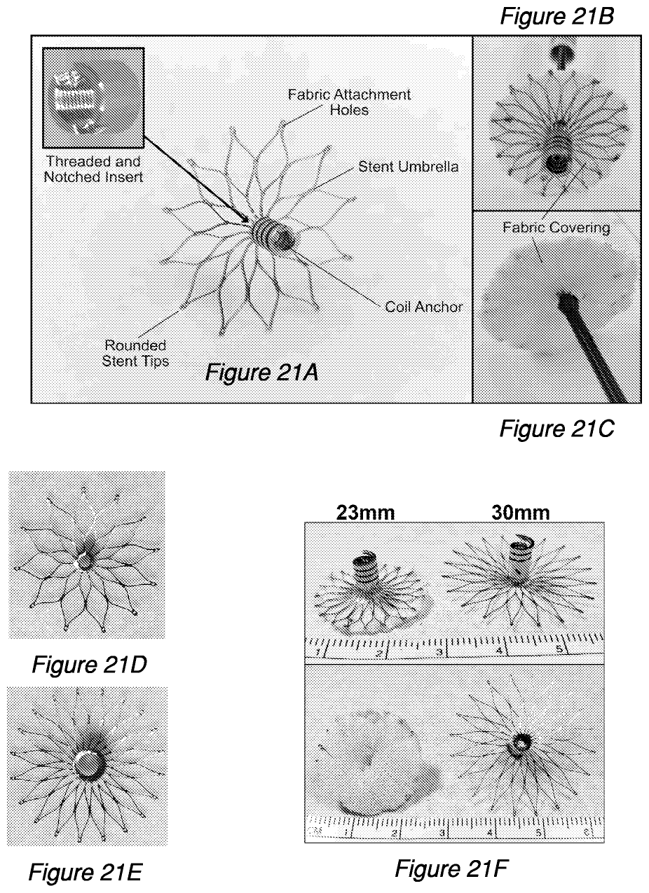

FIGS. 21A-21F are photographs of non-limiting samples of occluders usable in some embodiments. These samples generally correspond with FIGS. 4A, 4B, and 4C or else with FIG. 4D.

Compared with prior occluders, exemplary occluders disclosed herein may have reduced overall diameter in the collapsed state and in the anchor anchor profile. For instance, FIG. 21E shows an older 4.6 mm diameter anchor, whereas FIG. 21D shows a 2.8 mm diameter anchor Exemplary coil anchors have a profile/diameter of 1-3 mm in diameter, for example. This reduction in diameter allows the collapsible occluder to be used in a smaller sized delivery tool, making vascular access and device implantation easier compared with larger diameter anchors. A delivery sheath size of 12 Fr (4 mm diameter) or smaller may be used instead of a larger size such as 16 Fr (5.33 mm). At the top left of FIG. 21A is an enlarged portrayal of a threaded and notched insert (corresponding with FIG. 6A) which is fixed during manufacture inside an end of the anchor and/or between the anchor and the stent umbrella. Rounded stent umbrella tips are also shown in FIG. 21A. Tips which are not rounded (or not sufficiently rounded or dulled) risk perforating a fabric covering, shown in FIGS. 21B and 21C. Such perforation adds risk of potential tissue injury.

Where a range of values is provided in this disclosure, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. It should also be appreciated that indication of a rotation direction of “clockwise” may be replaced with “counterclockwise”, and “counterclockwise” with “clockwise”. Generally such a difference may involve only a change in the direction of threading of one or more components in one embodiment versus another embodiment.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible. Alternative methods may combine different elements of specific detailed methods described above and in the figures.

While exemplary embodiments of the present invention have been disclosed herein, one skilled in the art will recognize that various changes and modifications may be made without departing from the scope of the invention as defined by the appended claims.

1-61. (canceled)

62. An occluder for a left atrial appendage (LAA) of a heart, comprising:
a collapsible stent umbrella at a first end;
an anchor at second end;
and at least one of
a pair of moveable jaws on opposite sides of the anchor, sized to capture tissue of a wall of the LAA and permit the anchor to be secured to the captured tissue, and
a longitudinal opening at a center of the stent umbrella and one or more notches which open in a direction of the first end.

63. The occluder of claim 62, wherein the one or more notches includes at least two notches positioned on opposite sides of the longitudinal opening.

64. The occluder of claim 62, wherein the anchor has a diameter of 1-3 mm.

65. The occluder of claim 62, further comprising a longitudinally oriented pass through opening sized to permit guide wire insertion, tracking, and removal.

66. The occluder of claim 62, wherein the anchor is in a form of a helical coil, or in a form of a hook.

67. The occluder of claim 62, wherein the pair of moveable jaws are made from super-elastic or shape memory metal or metal alloy.

68. The occluder of claim 62, further comprising an insert positioned at a center of the stent umbrella, wherein the pair of moveable jaws are secured to the insert.

69. An occluder as recited in claim 62 wherein the collapsible stent umbrella comprises
a lattice framework formed from a super-elastic or shape memory metal or metal alloy which is annealed and quenched so as to set the lattice framework in a heat set deployed shape, wherein the lattice framework is deformable and returnable to the deployed shape via material super-elasticity, or wherein at body temperature the lattice framework assumes the heat set deployed shape in an absence of restricting external forces via material shape memory, wherein at freezing or near freezing temperatures the lattice framework is collapsible to a tubular shape, and wherein the lattice framework is sized to extend over an LAA ostium of a heart; and
a fabric covering affixed to the lattice framework; and wherein the anchor is
a coil anchor secured to or integrally formed with the lattice framework, wherein the coil anchor is a helical coil with a sharpened end opposite the lattice framework.

70. The collapsible occluder of claim 69, wherein the fabric covering is or has at least one of:
is a woven polyethylene terephthalate which is structured to facilitate tissue in-growth and encapsulation;
has attachment holes on lattice members at a circumferential periphery of the stent umbrella, and wherein the fabric covering is secured to the attachment holes;
is an expanded polytetrafluoroethylene (ePTFE) or other medical fabric material which is structured to facilitate tissue in-growth and encapsulation;
has attachment holes on lattice members at a circumferential periphery of the lattice framework, and wherein the fabric covering is secured to the attachment holes;
has rounded stent tips on lattice members at a circumferential periphery of the lattice framework; and
is attached directly to struts of the lattice framework.

71. The occluder of claim 69, wherein the coil anchor and the lattice framework are
integrally formed from a single piece of super-elastic or shape memory material, or
integrally formed from multiple pieces of super-elastic or shape memory material.

72. A method for occluding a left atrial appendage (LAA) of a heart, comprising:
positioning in the LAA an implant including an occluding portion and an anchoring portion, wherein the occluding portion has a collapsible lattice framework which is in a collapsed tubular shape during the positioning step;
grasping together a portion of an inside wall of LAA to produce a thickened tissue section;
anchoring the anchor portion to the thickened tissue section of the LAA; and
deploying the occluding portion in the left atrium so as to provide a seal between the left atrium and the LAA.

73. The method of claim 72 wherein the steps include
steering a catheter which delivers the occluder in a collapsed state to the LAA;
anchoring an anchor of the occluder to the inner wall of the LAA via a transfer of torque from a delivery tool to the anchor of the occluder;
deploying a lattice framework of the occluder to a deployed position in a left atrium of the heart and covering an opening to the LAA; and
releasing the delivery tool from the occluder.

74. A method for occluding a left atrial appendage (LAA) of a heart, comprising:
positioning a sheath with an occluder therein at the LAA, wherein the occluder comprises
a collapsible stent umbrella at a first end,
an anchor at a second end,
a threaded longitudinal opening at a center of the collapsible stent umbrella, and
one or more notches which open in a direction of the first end;
securing the anchor of the occluder to an inside wall of the LAA;
while holding the occluder stationary using a holding rod that interfaces with the one or more notches on the occluder, retracting the sheath from the occluder to deploy the collapsible stent umbrella to a deployed position in a left atrium of the heart and covering an opening to the LAA; and
unscrewing a threaded rod from the threaded longitudinal opening to leave the occluder in the LAA.

75. A control handle for a catheter delivery tool for installing an occluder in a left atrial appendage (LAA) of a heart, wherein the occluder comprises a collapsible stent umbrella at a first end, an anchor at a second end, a threaded longitudinal opening at a center of the stent umbrella, and one or more notches which open in a direction of the first end; the control handle comprising:
a primary handle;
a steerable catheter handle connected to the primary handle; and
one or more actuators for
steering a catheter which delivers the occluder to the LAA,
anchoring the anchor to an inner wall of the LAA,
deploying the collapsible stent umbrella to a deployed position in a left atrium of the heart and covering an opening to the LAA, and
releasing the catheter delivery tool from the occluder once the occluder is installed at the LAA.

76. A surgical system for occluding a left atrial appendage (LAA) of a heart, comprising:
an implant comprising
a collapsible lattice framework,
an anchor, and
a first interface; and
a delivery tool for implanting the implant in the heart, the delivery tool comprising
a second interface configured for holding the implant such that the implant is pushable and pullable and for transferring torque to the first interface to secure the anchor in tissue of the LAA, and
a sheath in which the implant is positionable in a collapsed state, and
one or more actuators for deploying the implant from a distal end of the sheath.

77. A delivery tool for surgical implantation of an implant, comprising:
an interface configured for holding the implant such that the implant is pushable and pullable and for transferring torque to the implant;
a sheath in which the implant is positionable in a collapsed state; and
one or more actuators for deploying the implant from a distal end of the sheath,
wherein the sheath and the interface are moveable relative to one another such that the interface is moveable between a first position inside the sheath to a second position outside the sheath.
